(19) United States
(12) Patent Application Publication
(10) Pub. No.: US 2025/0144981 A1
(43) Pub. Date: May 8, 2025

(54) Fragrancing System for Fragrancing an Interior of a Motor Vehicle

(71) Applicant: Bayerische Motoren Werke Aktiengesellschaft, Muenchen (DE)

(72) Inventor: Annabelle COFFINET, Muenchen (DE)

(21) Appl. No.: 18/833,458

(22) PCT Filed: Mar. 24, 2023
(86) PCT No.: PCT/EP2023/057714

§ 371 Date: Jul. 26, 2024

(30) Foreign Application Priority Data
Apr. 4, 2022 (DE) 10 2022 108 042.0

(51) Int. Cl. B60H 3/00; B62D 1/04

(52) U.S. Cl. CPC B60H 3/0014; B62D 1/04

(57) ABSTRACT

A fragrancing system for fragrancing an interior of a motor vehicle is provided. The interior of the vehicle has at least one interior part which is movable such that, when transformed from a first condition to a second condition, it at least temporarily exposes a cavity, and the transformation produces an air flow in the cavity; and a fragrance support is arranged such in the cavity that it is activated by the air flow and fragrance molecules are emitted toward the exterior of the cavity.

### BACKGROUND AND SUMMARY

The invention relates to a fragrancing system for fragrancing an interior of a vehicle.

There are already fragrancers in vehicles focusing on disseminating a pleasant fragrance in the interior of the vehicle. There are already systems here which discharge a fragrance via the air conditioning system of the vehicle. Passive fragrancing systems which permanently dispense a fragrance are also already known.

There are, however, still improvements required in the field of fragrancing the interior of a vehicle.

It is, therefore, an object of this invention to provide an improved fragrancing system for fragrancing an interior of a vehicle. This object is achieved according to the claimed invention.

Proposed is a fragrancing system for fragrancing an interior of a vehicle, wherein at least one interior part which is movable in such a manner that, when transformed from a first state to a second state, it at least briefly releases a cavity is present in the interior of the vehicle, and wherein an airflow in the cavity is generated by the transformation, wherein a fragrance carrier is disposed in the cavity in such a manner that the former is activated by the airflow and dispenses fragrance molecules to the exterior of the cavity.

In one design embodiment it is provided that furthermore a chamber in which the fragrance carrier is placed is provided in the cavity, wherein the chamber is formed in such a manner that the latter in the first and the second state is closed on all sides, and during transformation at least briefly releases an opening in the direction of the interior of the vehicle in such a manner that the airflow enters and flows over the fragrance carrier, activates the latter, and the dispensed fragrance molecules flow to the exterior of the chamber and of the cavity.

In one design embodiment it is provided that the region of the chamber, which during transformation at least briefly releases an opening in the direction of the interior of the vehicle, in the first and/or the second state is covered by the interior part, or has a cover which is opened during transformation.

In one design embodiment it is provided that the interior part is a steering wheel which can transform in terms of its shape.

In one design embodiment it is provided that the interior part is a steering wheel, a headrest, an entertainment device, a support in the region of a seat, and wherein the second state is an altered position and/or a position which is visible to and/or utilizable by an occupant, or a position which is no longer visible to and/or utilizable by the occupant.

In one design embodiment it is provided that the fragrance carrier is a solid substance having fragrance molecules which are located therein and activatable by the airflow, and comprising at least one polymer, or wherein the fragrance carrier is a material impregnated with a fragrance.

In one design embodiment it is provided that the chamber is formed in such a manner that the fragrance carrier disposed therein can be replaced or refilled.

Furthermore proposed is a vehicle having the fragrancing system for fragrancing an interior of the vehicle. The vehicle is advantageously a fully automated or self-driving vehicle.

In one design embodiment it is provided that the vehicle additionally has one or a plurality of devices which are specified to visually and/or acoustically orchestrate the dispensing of fragrance molecules provided by the fragrancing system according to defined criteria.

Further features and advantages of the invention are derived from the description hereunder of exemplary embodiments of the invention by way of the drawings which show details according to the invention, and from the claims. The individual features can in each case be implemented individually or in multiples in any combination in a variant of the invention.

Preferred embodiments of the invention will be explained in more detail hereunder by way of the appended drawings.

### BRIEF DESCRIPTION OF THE DRAWINGS

FIG. 1 shows a schematic illustration of a steering wheel in a first state.

FIG. 2 shows a schematic illustration of a steering wheel during transformation, and the fragrancing system according to one embodiment of the present invention.

FIG. 3 shows a schematic illustration of a steering wheel in a second state.

### DETAILED DESCRIPTION OF THE DRAWINGS

Identical elements or functions are provided with the same reference signs in the description of the figures hereunder.

The proposed fragrancing system serves to provide brief fragrancing of part of an interior of a vehicle. The brief fragrancing takes place while an interior part 1 of the vehicle transforms from a first to a second state.

The transformation of interior parts (only a steering wheel 1 is shown as an interior part in the figure) is possible in particular in vehicles of automation level 4 and above (fully automated driving). As from this level, (specific) driving tasks can be delegated, and the driver can attend to other matters.

In this way, the steering wheel 1 can be retracted into the dashboard 2, for example, as is schematically illustrated in FIG. 2, or be deployed from the latter again. Also, a seat can be rotated, and the headrest and other supports such as arm rests and/or footrests can be altered in comparison to the positioning up to now. A transformation thus takes place in which either the position of the interior part is altered, this potentially being the case for example in a transformation of a headrest or any other support, or the interior part moves from a position which is visible to and/or utilizable by an occupant to a position no longer visible to and/or utilizable by the occupant.

A transformation of the interior part per se may also take place. For example, the steering wheel 1 can undergo a geometric transformation, i.e. assume a different shape.

In both types of transformation, at least one cavity 20 which briefly opens in the direction of the interior of the vehicle is created during the transformation from the first to the second state. Otherwise, this cavity 20 is covered by the interior part (by the entire interior part as well as only by parts thereof).

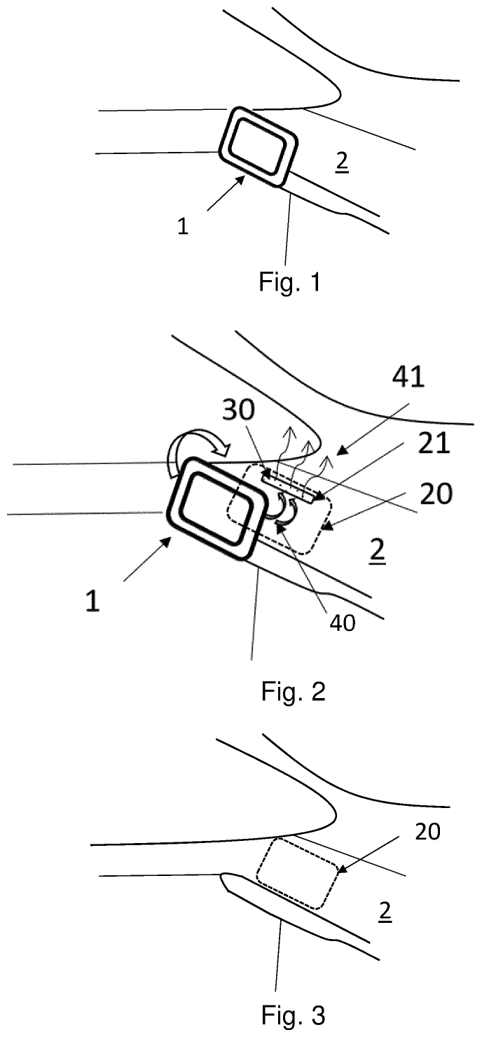

In the context of the description, a state prior to transformation, as shown in FIG. 1, in which the steering wheel 1 is visible to and utilizable by an occupant, is referred to as the first state, and a state after transformation, as shown in FIG. 3, in which the steering wheel 1 is no longer visible to and/or utilizable by an occupant, is referred to as the second state. Alternatively, the second state is a state in which only the position or shape of the interior part has changed.

The cavity 20 created during transformation can be utilized for initiating a brief fragrancing of the interior. For this purpose, a fragrance carrier 30 can be placed in the cavity 20 in such a manner that the fragrance carrier 30 is activated by the air draft 40 created by virtue of the transformation (as illustrated by arrows in FIG. 2), i.e. that the fragrance carrier 30 releases fragrance molecules by virtue of the air draft 40. The fragrance carrier 30 herein is to be disposed in the cavity 20 in such a way that the released fragrance molecules flow into the interior, ideally in the direction of the occupant, by virtue of the air draft 40. The air draft 40 as well as the fragrance flow 41 are identified by arrows 40, 41 in FIG. 2.

Furthermore indicated in FIG. 2 is a chamber 21 which is disposed in the cavity 20. This chamber 21 can be provided if the cavity 20 per se does not form a chamber, for example opens into a region that does not point toward the interior, for example behind the dashboard 2. It would be possible here that fragrance molecules which are released even without transformation, for example by virtue of temperature fluctuations, are unnecessarily wasted. For this reason, a chamber 21 which forms a closed space, thus is closed on all sides, except during transformation, can be provided. This space herein is formed in such a way that fragrance molecules which are released without transformation, for example by virtue of temperature fluctuations, are retained in the chamber 21 and released only during transformation. This means that the chamber 21 either is covered (entirely or partially) by the interior part in the region pointing toward the interior, or has a dedicated cover which is opened during transformation. This can take place conjointly with the interior part, or the cover may be separately actuated. During transformation, the fragrance molecules can then (and only then) flow into the interior of the vehicle (fragrance flow 41).

The fragrance carrier 30 can be a solid substance having fragrance molecules which are located therein and activatable by an airflow 40. Polymers are particularly suitable herein. However, the fragrance carrier 30 can also be a material impregnated with a fragrance.

In one embodiment the fragrance carrier 30 is disposed in such a manner that the latter can be replaced or refilled. This can be enabled in that, for example, the transformation can be halted once the cavity 20 is visible. This can be performed by a workshop, for example, so that the fragrance carrier 30 can also be replaced, or fragrance refilled, during a service.

The proposed fragrancing system is a passive fragrancing system, i.e. only the airflow 40 is required in order to release fragrance molecules.

The direction in which the airflow 40 flows into the cavity 20, or the chamber 21, and in which the airflow 41 flows out of the cavity 20, or the chamber 21, depends on many factors, for example on the shape of the cavity 20, or of the chamber 21, on the shape and/or type of the fragrance carrier 30, on the placing of the fragrance carrier 30, or else on the dynamics and the direction of the transformation. An optimal airflow and fragrance flow 40, 41 can therefore be determined, for example by way of a flow simulation, based on all factors that have an influence thereon.

The following interior parts can advantageously be used for fragrancing the interior, as long as they can be transformed: steering wheel, headrest, entertainment device, support in the region of a seat. Such interior parts that are located close to the head of an occupant are particularly advantageous because the occupant can better experience the fragrance.

The transformation, and thus also the fragrance flow 41, can also be accompanied by a visual and/or acoustic orchestration if the vehicle has a corresponding equipment with visual and/or acoustic devices and a corresponding actuation.

The vehicle is advantageously a fully automated or self-driving vehicle.

### LIST OF REFERENCE SIGNS

1 Steering wheel (interior part)
2 Dashboard
20 Cavity in 2
21 Chamber in 20
30 Fragrance carrier
40 Airflow to 21
41 Fragrance flow from 30

1.-10. (canceled)

11. A fragrancing system for fragrancing an interior of a vehicle, the fragrancing system comprising:
an interior part which is movable such that, when transformed from a first state to a second state, the interior part at least briefly releases a cavity present in an interior of the vehicle, wherein an airflow in the cavity is generated by the transformation; and
a fragrance carrier disposed in the cavity such that fragrance carrier is activated by the airflow and dispenses fragrance molecules to an exterior of the cavity.

12. The fragrancing system according to claim 11, wherein:
a chamber in which the fragrance carrier is placed is provided in the cavity, and
the chamber is formed such that the chamber in the first state and the second state is closed on all sides, and during the transformation at least briefly releases an opening in a direction of the interior of the vehicle such that the airflow enters and flows over the fragrance carrier, activates the fragrance carrier, and the dispensed fragrance molecules flow to an exterior of the chamber and to the exterior of the cavity.

13. The fragrancing system according to claim 12, wherein a region of the chamber, which during the transformation at least briefly releases the opening in the direction of the interior of the vehicle, in the first state and/or the second state is covered by the interior part, or has a cover which is opened during the transformation.

14. The fragrancing system according to claim 11, wherein the interior part is a steering wheel that is transformable in terms of shape.

15. The fragrancing system according to claim 11, wherein:
the interior part is a steering wheel, a headrest, an entertainment device, or a support in a region of a seat,
the second state is an altered position and/or a position which is visible to and/or utilizable by an occupant, or a position which is no longer visible to and/or utilizable by the occupant.

16. The fragrancing system according to claim 11, wherein:
the fragrance carrier is a solid substance having fragrance modules which are located in the fragrance carrier and are activatable by the airflow, and comprise at least one polymer, or
the fragrance carrier is a material impregnated with a fragrance.

17. The fragrancing system according to claim 11, wherein the fragrance carrier is disposed such that the fragrance carrier is replaceable or refillable.

18. A vehicle comprising the fragrancing system according to claim 11.

19. The vehicle according to claim 18, wherein the vehicle is a fully automated or a self-driving vehicle.

20. The vehicle according to claim 18, wherein the vehicle comprises a device which is configured to visually and/or acoustically orchestrate dispensing of the fragrance molecules provided by the fragrancing system according to defined criteria.
